(19) United States
(12) Patent Application Publication
(10) Pub. No.: US 2026/0136837 A1
(43) Pub. Date: May 14, 2026

(54) DEGRADABLE ULTRASOUND TRANSDUCER

(71) Applicant: KOREA UNIVERSITY RESEARCH AND BUSINESS FOUNDATION, Seoul (KR)

(72) Inventors: Jung-Yeol YEOM, Seoul (KR); Jahyun KOO, Seoul (KR); Seung-Kyun KANG, Seoul (KR); Gi-heon KIM, Seoul (KR)

(73) Assignee: KOREA UNIVERSITY RESEARCH AND BUSINESS FOUNDATION, Seoul (KR)

(21) Appl. No.: 18/686,163

(22) PCT Filed: Aug. 24, 2022
(86) PCT No.: PCT/KR2022/012664

§ 371 Date: Feb. 23, 2024

(30) Foreign Application Priority Data
Aug. 25, 2021 (KR) 10-2021-0112039
Aug. 22, 2022 (KR) 10-2022-0104604

(51) Int. Cl. H10N 30/20; A61L 31/02; A61L 31/06; B06B 1/06; H10N 30/853; H10N 30/87

(52) U.S. Cl. CPC H10N 30/20; A61L 31/022; A61L 31/06; H10N 30/853; H10N 30/87; B06B 1/06

(57) ABSTRACT

The present invention relates to a degradable ultrasound transducer which is naturally degraded in a use environment, wherein a degradable ultrasound transducer according to an embodiment of the present invention comprises: a piezoelectric layer (10) including a degradable piezoelectric element; a first degradable electrode (20) disposed on one side of the piezoelectric layer (10); and a second degradable electrode (30) disposed on the other side of the piezoelectric layer (10).

### TECHNICAL FIELD

The present invention relates to a degradable ultrasonic transducer, and more specifically to a degradable ultrasonic transducer that is naturally degraded in a use environment.

### BACKGROUND ART

Ultrasonic transducers refer to devices that convert electrical signals into ultrasonic signals (converse piezoelectric effect) and convert physical signals into electrical signals (piezoelectric effect). Ultrasonic transducers are being increasingly used across all industries, including medical devices and displays. Piezoelectric materials are mainly used for ultrasonic transducers.

PZT-based piezoelectric materials (Pb[ZrₓTi₁₋ₓ]O₃) for ultrasonic transducers are used in various industrial fields due to their high piezoelectric efficiency but are pointed out as causes of environmental pollution due to the presence of toxic lead (Pb). In attempts to solve these problems, eco-friendly lead-free piezoelectric materials have been developed. However, the low piezoelectric efficiency of these piezoelectric materials compared to PZT needs to be solved. Furthermore, since the piezoelectric materials cannot be recycled immediately after use, they release toxic materials when electronic devices are discarded, which may become the subject of ever-increasing environmental regulations.

Under these circumstances, there arises a need to develop piezoelectric materials that are degraded in nature. Some previous studies have revealed that the use of polymer-based piezoelectric materials has the potential to develop degradable ultrasonic piezoelectric transducers, but the low piezoelectric efficiency of polymer-based piezoelectric materials limits their industrial application. As the market for implantable medical devices has recently expanded, considerable research has focused on piezoelectric nanogenerators (PENGs) for power supply to implanted medical devices. Biocompatibility should be taken into consideration for applying piezoelectric materials to implantable medical devices. For example, a focused ultrasonic transducer may generate low-intensity focused ultrasound that temporarily opens brain-blood barrier (BBB) to enhance the drug delivery effect or may be utilized for neuromodulation. Alternatively, a focused ultrasonic transducer may generate high-intensity focused ultrasound that kills brain tumors. In this case, focused ultrasound is implanted into the brain tissue of a patient with brain disease for effective drug delivery to the brain tissue. However, the implantation into the brain tissue puts the patient at risk, particularly removal of the implanted medical device after treatment of the brain disease may cause more serious secondary damage to the brain tissue. If the medical device is left in the brain tissue, an immune reaction may occur that makes the lesion worse.

Thus, there is an urgent need for a solution to the problems of conventional ultrasonic transducers.

### DETAILED DESCRIPTION OF THE INVENTION

### Problems to be Solved by the Invention

The present invention has been made in an effort to solve the problems of the prior art, and one aspect of the present invention is to provide an ultrasonic transducer that possesses high piezoelectric efficiency, is naturally degradable in a use environment, and has a controllable degradation rate.

### Means for Solving the Problems

A degradable ultrasonic transducer according to an embodiment of the present invention includes a piezoelectric layer including a degradable piezoelectric material, a first degradable electrode arranged at one side of the piezoelectric layer, and a second degradable electrode arranged at the other side of the piezoelectric layer.

According to an exemplary embodiment of the present invention, the piezoelectric material may include at least one selected from the group consisting of Rochelle salt, potassium dihydrogen phosphate (KDP), ammonium dihydrogen phosphate (ADP), and triglycine sulfate (TGS).

According to an exemplary embodiment of the present invention, the piezoelectric material may include at least one selected from the group consisting of zinc oxide (ZnO) and quartz.

According to an exemplary embodiment of the present invention, the piezoelectric material may be in the form of one or more nanostructures selected from the group consisting of nanorods, nanopillars, and nanowires.

According to an exemplary embodiment of the present invention, each of the first and second electrodes may independently include at least one material selected from the group consisting of zinc (Zn), zinc (Zn)-magnesium (Mg) alloys, magnesium (Mg), molybdenum (Mo), and tungsten (W).

According to an exemplary embodiment of the present invention, the degradable ultrasonic transducer may further include a degradable cover layer surrounding the piezoelectric layer.

According to an exemplary embodiment of the invention, the cover layer may include at least one selected from the group consisting of poly(lactic-co-glycolic acid) (PLGA) and poly-butanedithiol pentenoic anhydride (PBTPA).

According to an exemplary embodiment of the present invention, the degradable ultrasonic transducer may further include a degradable matching layer arranged to face the piezoelectric layer with the first electrode interposed therebetween.

According to an exemplary embodiment of the present invention, the matching layer may include at least one selected from the group consisting of silicon dioxide (SiO₂), zinc oxide (ZnO), and poly(lactic-co-glycolic acid) (PLGA).

According to an exemplary embodiment of the present invention, the degradable ultrasonic transducer may further include a degradable backing layer arranged to face the piezoelectric layer with the second electrode interposed therebetween.

According to an exemplary embodiment of the present invention, the backing layer may include at least one metal powder selected from the group consisting of zinc (Zn) powder, magnesium (Mg) powder, and tungsten (W) powder and at least one material selected from the group consisting of natural wax mixtures, poly(lactic-co-glycolic acid) (PLGA), poly-butanedithiol pentenoic anhydride (PBTPA), titanium (Ti), alumina, ceramics, and animal bones.

The features and advantages of the present invention will become apparent from the following description with reference to the accompanying drawings.

Prior to the detailed description of the invention, it should be understood that the terms and words used in the specification and the claims are not to be construed as having common and dictionary meanings but are construed as having meanings and concepts corresponding to the technical spirit of the present invention in view of the principle that the inventor can define properly the concept of the terms and words in order to describe his/her invention with the best method.

### Effects of the Invention

The specific kind and structure of the piezoelectric material used in the degradable ultrasonic transducer of the present invention ensures high piezoelectric efficiency of the ultrasonic transducer, allows the ultrasonic transducer to be naturally degraded in a use environment, and enables degradation of the ultrasonic transducer at a controlled rate. Therefore, the ultrasonic transducer of the present invention can be applied to eco-friendly electronic devices.

In addition, due to its bioabsorbability, the degradable ultrasonic transducer of the present invention can be applied to implantable medical devices where a high degree of biocompatibility is required.

### BRIEF DESCRIPTION OF THE DRAWINGS

FIG. 1 is a cross-sectional view of a degradable ultrasonic transducer according to one embodiment of the present invention.

FIG. 2 is a cross-sectional view of a degradable ultrasonic transducer according to a further embodiment of the present invention.

FIG. 3 is a cross-sectional view of a degradable ultrasonic transducer according to another embodiment of the present invention.

FIG. 4 is an image of a Rochelle salt crystal grown in Experimental Example 1.

FIG. 5 is an image of a potassium dihydrogen phosphate crystal grown in Experimental Example 2.

FIG. 6 is a side view of an ultrasonic transducer fabricated in Experimental Example 3.

FIG. 7 is an image showing an experimental setting for testing the performance of ultrasonic transducers in Experimental Example 4.

FIG. 8 shows data verifying the piezoelectric effect of an ultrasonic transducer using a Rochelle salt crystal as a piezoelectric element in Evaluation Example 1.

FIG. 9 shows data verifying the piezoelectric effect of an ultrasonic transducer using a zinc oxide crystal as a piezoelectric element in Evaluation Example 1.

FIG. 10 shows the results of simulations for ultrasound generation from ultrasonic transducers using zinc oxide crystals as piezoelectric elements in Evaluation Example 2.

FIG. 11 shows the experimental results confirming the ranges where ultrasonic waves were generated from ultrasonic transducers with different thicknesses using zinc oxide as a piezoelectric element in Evaluation Example 3.

### BEST MODE FOR CARRYING OUT THE INVENTION

The objects, specific advantages, and novel features of the present invention will become apparent from the following detailed description and preferred embodiments in conjunction with the accompanying drawings. It should be noted that in the drawings, the same components are denoted by the same reference numerals even though they are depicted in different drawings. Although such terms as “first” and “second,” etc. may be used to describe various components, these components should not be limited by above terms. These terms are used only to distinguish one component from another. In the description of the present invention, detailed explanations of related art are omitted when it is deemed that they may unnecessarily obscure the essence of the present invention.

Preferred embodiments of the present invention will now be described in detail with reference to the accompanying drawings.

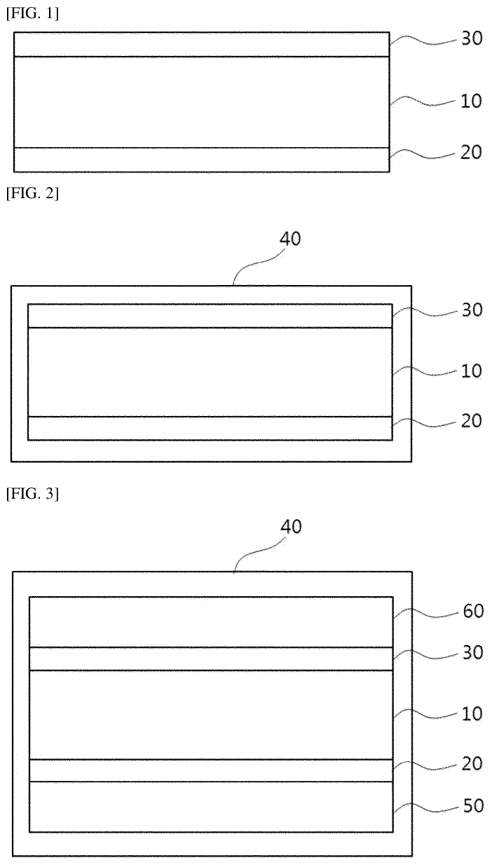

FIG. 1 is a cross-sectional view of a degradable ultrasonic transducer according to one embodiment of the present invention.

As illustrated in FIG. 1, the degradable ultrasonic transducer includes a piezoelectric layer 10 including a degradable piezoelectric material, a first degradable electrode 20 arranged at one side of the piezoelectric layer 10, and a second degradable electrode 30 arranged at the other side of the piezoelectric layer 10.

Ultrasonic transducers refer to devices that convert electrical signals into ultrasonic signals (converse piezoelectric effect) and convert physical signals into electrical signals (piezoelectric effect). Ultrasonic transducers are being increasingly used across all industries. As research on the development of electronic devices, displays, energy systems, and medical devices using ultrasonic waves increases, the need for high efficiency piezoelectric elements increases. However, the presence of lead in conventional piezoelectric elements causes serious problems, including environmental pollution and biotoxicity. There also arises a need for biodegradable eco-friendly electronic devices due to the flood of electronic waste. In attempts to solve the problems of lead-containing piezoelectric elements, eco-friendly lead-free piezoelectric elements and polymer-based piezoelectric elements have been investigated. However, these piezoelectric elements are limited in commercialization because of their low efficiency. The present invention has been devised to overcome this limitation.

As described above, the degradable ultrasonic transducer includes a piezoelectric layer 10, a first electrode 20, and a second electrode 30.

The piezoelectric layer 10 converts applied electrical signals into mechanical vibrations to generate ultrasonic waves. The piezoelectric layer 10 includes a piezoelectric material. The term “piezoelectric material” refers to a material that has a piezoelectric effect in which a voltage is generated in response to a mechanical pressure applied and a converse piezoelectric effect in which a mechanical deformation is induced by an applied voltage. The piezoelectric material used in the present invention is degradable as well as having both piezoelectric and converse piezoelectric effects. Particularly, the degradability of the piezoelectric material makes the piezoelectric layer 10 degradable. The degradable piezoelectric material is hygroscopic and soluble in water and other solvents, ensuring its natural degradation in a use environment. The degradable piezoelectric material may be a biodegradable one that is degraded by bacteria, fungi, and other organisms. The degradable piezoelectric material may also be a bioabsorbable one that is absorbed and degraded in vivo. The bioabsorbable piezoelectric material can be applied to implantable medical devices.

For example, the piezoelectric material may include a first degradable piezoelectric material and/or a second degradable piezoelectric material. The first degradable piezoelectric material may include at least one material selected from the group consisting of Rochelle salt, potassium phosphate crystals, and triglycine sulfate (TGS). The term “potassium phosphate crystals” is used as a generic name for all potassium phosphate-based compounds, for example, potassium dihydrogen phosphate (KDP) and ammonium dihydrogen phosphate (ADP). The first degradable piezoelectric material corresponds to a piezoelectric material that is relatively hygroscopic and highly soluble. When applied to humans, the first degradable piezoelectric material can be selected from Rochelle salt, potassium dihydrogen phosphate (KDP), and triglycine sulfate (TGS) that are less toxic to humans. Particularly, the first degradable piezoelectric material is in the form of a salt crystal that has high piezoelectric efficiency and is readily degradable.

The second degradable piezoelectric material may include at least one selected from the group consisting of zinc oxide (ZnO) and quartz. The second degradable piezoelectric material is less hygroscopic and soluble than the first degradable piezoelectric material. Thus, the second degradable piezoelectric material may be in the form of nanostructures to accelerate the degradation rate and improve the piezoelectric efficiency. The nanostructures may be selected from the group consisting of nanorods, nanopillars, nanowires, and combinations of two or more thereof. That is, the second degradable piezoelectric material may be in the form of single or multiple nanostructures.

However, the first and second degradable piezoelectric materials are not necessarily limited to those exemplified above and are not particularly limited as long as they are degradable. The degradation rate of the piezoelectric layer 10 may be adjusted depending on the thickness of the piezoelectric layer 10 or the kind and shape of the piezoelectric material.

The first electrode 20 is arranged at one side of the piezoelectric layer 10 and is degradable. The second electrode 30 is arranged at the other side of the piezoelectric layer 10 and is degradable. Each of the first electrode 20 and the second electrode 30 may independently be made of a conductive material that is hygroscopic, soluble, biodegradable, and bioabsorbable and can be easily degraded. The conductive material may be coated on one side of the piezoelectric layer 10 to form the first electrode 20 and coated on the other side of the piezoelectric layer 10 to form the second electrode 30. The conductive material may be coated to appropriate thicknesses on both surfaces of the piezoelectric layer 10 using a suitable device such as an E-beam evaporator or sputter.

One of the first electrode 20 and the second electrode 30 functions as an anode (or signal electrode) of the piezoelectric layer 10 and the other electrode functions as a cathode (or ground electrode) of the piezoelectric layer 10.

Each of the first 20 and second degradable electrodes 30 may independently include at least one material selected from the group consisting of zinc (Zn), zinc (Zn)-magnesium (Mg) alloys, magnesium (Mg), molybdenum (Mo), and tungsten (W). The first electrode 20 and the second electrode 30 do not necessarily have to be made of the same material and may be made of different materials. Any conductive and degradable materials may be used for the first electrode 20 and the second electrode 30.

FIG. 2 is a cross-sectional view of a degradable ultrasonic transducer according to a further embodiment of the present invention.

Referring to FIG. 2, the degradable ultrasonic transducer further includes a degradable cover layer 40.

The cover layer 40 surrounds and covers the piezoelectric layer 10. The cover layer 40 may surround both the first electrode 20 and the second electrode 30 as well as the piezoelectric layer 10. That is, the cover layer 40 encapsulates the piezoelectric layer 10 or the first electrode 20/piezoelectric layer 10/second electrode 30 structure. The cover layer 40 is composed of a degradable packaging material and surrounds the periphery of the piezoelectric layer 10 or the first electrode 20/piezoelectric layer 10/second electrode 30 structure to block the ingress of moisture, and as a result, the internal components can be protected from moisture. In addition, since the degradation time of the cover layer is controlled depending on the thickness of the cover layer, the degradation rates (including biodegradation and bioabsorption rates) of the internal components can be adjusted by the cover layer.

The cover layer 40 may be composed of a hydrophobic polymer. For example, the cover layer 40 may include at least one selected from the group consisting of, but not necessarily limited to, poly(lactic-co-glycolic acid) (PLGA) and poly-butanedithiol pentenoic anhydride (PBTPA).

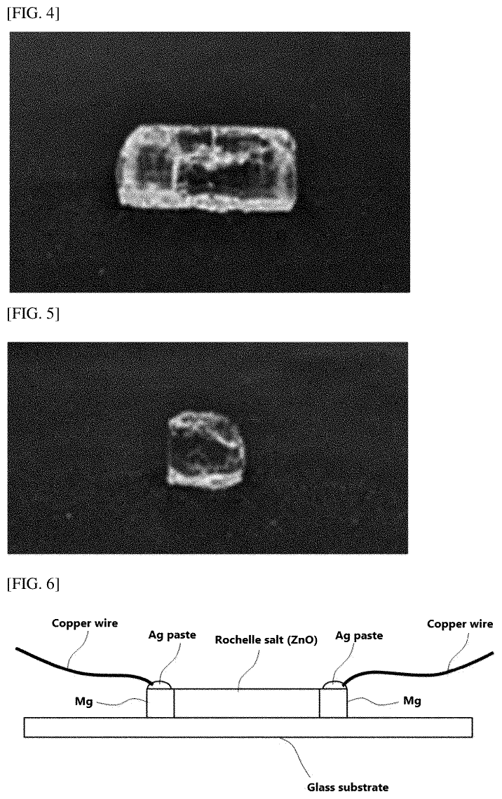

FIG. 3 is a cross-sectional view of a degradable ultrasonic transducer according to another embodiment of the present invention.

As illustrated in FIG. 3, the degradable ultrasonic transducer further includes a matching layer.

The matching layer 50 appropriately matches the acoustic impedances of the piezoelectric layer 10 and a target to transmit ultrasonic waves to the target or reduce loss of ultrasonic waves received from the target. The matching layer 50 may be arranged to face the piezoelectric layer 10 with the first electrode 20 interposed therebetween. The matching layer 50 is degradable. For example, the degradable matching layer 50 may include at least one selected from the group consisting of, but not necessarily limited to, silicon dioxide (SiO₂), zinc oxide (ZnO), and poly(lactic-co-glycolic acid) (PLGA).

The degradable ultrasonic transducer further includes a backing layer 60.

The backing layer 60 is arranged to face the piezoelectric layer 10 with the second electrode 30 interposed therebetween. The backing layer 60 absorbs ultrasonic waves propagating in directions other than the direction toward a target. The backing layer 60 is also degradable. For example, the degradable backing layer 60 may include at least one metal powder selected from the group consisting of zinc (Zn) powder, magnesium (Mg) powder, and tungsten (W) powder and at least one material selected from the group consisting of natural wax mixtures, poly(lactic-co-glycolic acid) (PLGA), poly-butanedithiol pentenoic anhydride (PBTPA), titanium (Ti), alumina, ceramics, and animal bones. Particularly, when the ultrasonic transducer of the present invention is used in a bioimplantable medical device, a hard tissue of the body capable of absorbing ultrasonic waves may be used as the backing layer 60. A bone such as a skull bone may be used as the backing layer 60. However, the material for the backing layer 60 is not necessarily limited to those exemplified above and is not particularly limited as long as it is degradable in a predetermined environment while absorbing ultrasonic waves.

Since ultrasonic waves generated in the piezoelectric layer 10 propagate spherically from the piezoelectric layer 10, there is a limitation in intensively delivering transmitting the ultrasonic waves to a target to be imaged or treated. This limitation can be overcome by the formation of the matching layer 50 and the backing layer 60.

Th cover layer 40 may completely surround and encapsulate the matching layer 50/first electrode 20/piezoelectric layer 10/second electrode 30/backing layer 60 structure.

Overall, the specific kind and structure of the piezoelectric material used in the degradable ultrasonic transducer of the present invention ensure high piezoelectric efficiency of the ultrasonic transducer, allows the ultrasonic transducer to be naturally degraded in a use environment, and enables degradation of the ultrasonic transducer at a controlled rate. Therefore, the ultrasonic transducer of the present invention can be applied to eco-friendly electronic devices. In addition, due to its bioabsorbability, the degradable ultrasonic transducer of the present invention can be applied to implantable medical devices where a high degree of biocompatibility is required.

### MODE FOR CARRYING OUT THE INVENTION

The present invention will be more specifically explained with reference to the following experimental examples.

### Experimental Example 1: Production of Piezoelectric Element Based on Rochelle Salt Crystal

First, potassium sodium tartrate tetrahydrate was mixed and triturated with deionized water (D.I. water). The powder-type Rochelle salt was subjected to repeated heating and cooling cycles to induce crystal growth. The reaction was allowed to proceed for a long time. The grown Rochelle salt crystal was cut to an appropriate thickness and size considering the desired frequency and power. Surface treatment was performed with a polisher to produce a piezoelectric element based on the Rochelle salt crystal. An image of the grown Rochelle salt crystal is shown in FIG. 4.

### Experimental Example 2: Production of Piezoelectric Element Based on Potassium Dihydrogen Phosphate Crystal

First, powder-type potassium dihydrogen phosphate was dissolved in deionized water (D.I. water) under heating. Then, a thread was immersed in the solution to induce crystallization of the potassium dihydrogen phosphate. The crystal was allowed to grow for a long time. The grown potassium dihydrogen phosphate crystal was cut to an appropriate thickness and size considering the desired frequency and power. Surface treatment was performed with a polisher to produce a piezoelectric element based on the potassium dihydrogen phosphate crystal. An image of the grown potassium dihydrogen phosphate crystal is shown in FIG. 5.

### Experimental Example 3: Fabrication of Degradable Ultrasonic Transducers

Magnesium was coated on both surfaces of each of the piezoelectric element based on the Rochelle salt crystal and the piezoelectric element based on the potassium dihydrogen phosphate crystal by sputtering to form first and second electrodes. Next, copper conductive wires were connected to the first and second electrodes through silver (Ag) pastes to fabricate an ultrasonic transducer. Then, the ultrasonic transducer was fixed onto a glass substrate using PI tapes to prevent short circuits between a medium used for subsequent ultrasound generation experiments and the conductive copper wires and between the medium and the electrodes and corrosion of the conductive copper wires and the electrodes. A side view of the ultrasonic transducer was shown in FIG. 6.

### Experimental Example 4: Experimental Setting

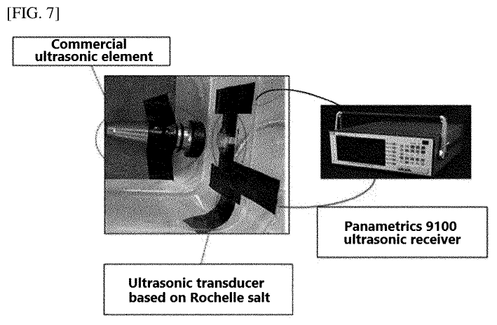

In this example, an experiment was conducted to verify whether the degradable ultrasonic transducers fabricated in Experimental Example 3 generated ultrasonic waves and had a piezoelectric effect. To this end, an experimental setting shown in FIG. 7 was designed to test the performance of the ultrasonic transducers. Since a piezoelectric effect occurs in a piezoelectric material that converts a voltage into ultrasonic waves and an external pressure into a voltage, an external pressure was applied to the ultrasonic transducer to generate electrical signals and the electrical signals were analyzed to verify the piezoelectric effect of the ultrasonic transducer.

For performance testing, the degradable ultrasonic transducer was connected to an ultrasonic receiver, and a commercial ultrasonic element and the degradable ultrasonic transducer were immersed in a medium (for example, water, ethanol or fat). Then, external ultrasonic waves were applied from the commercial ultrasonic element to the degradable ultrasonic transducer. To verify the piezoelectric effect, an analysis was performed to determine whether the degradable ultrasonic transducer converted the external pressure into electrical signals through the ultrasonic receiver.

### Evaluation Example 1: Verification of Ultrasound Generation from the Degradable Ultrasonic Transducers and Piezoelectric Effect of the Degradable Ultrasonic Transducers

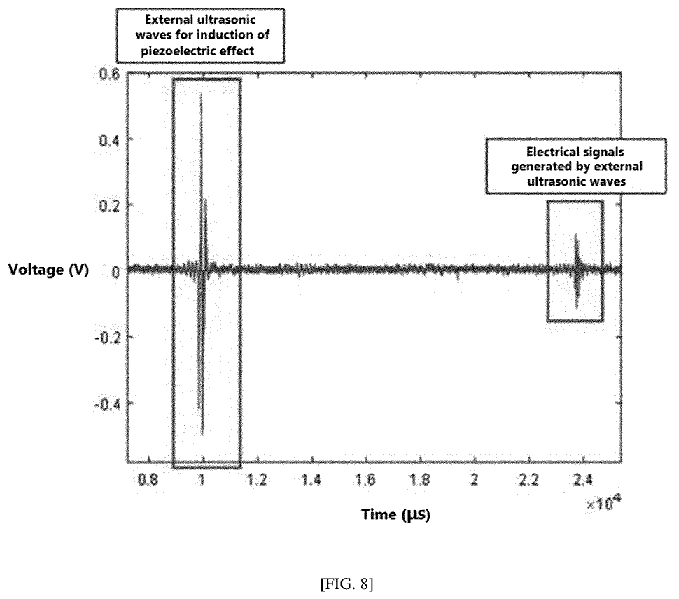

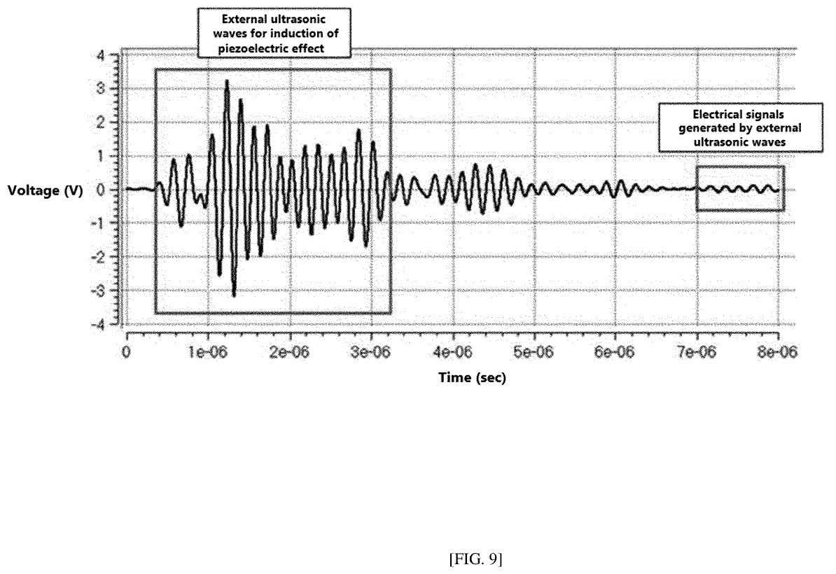

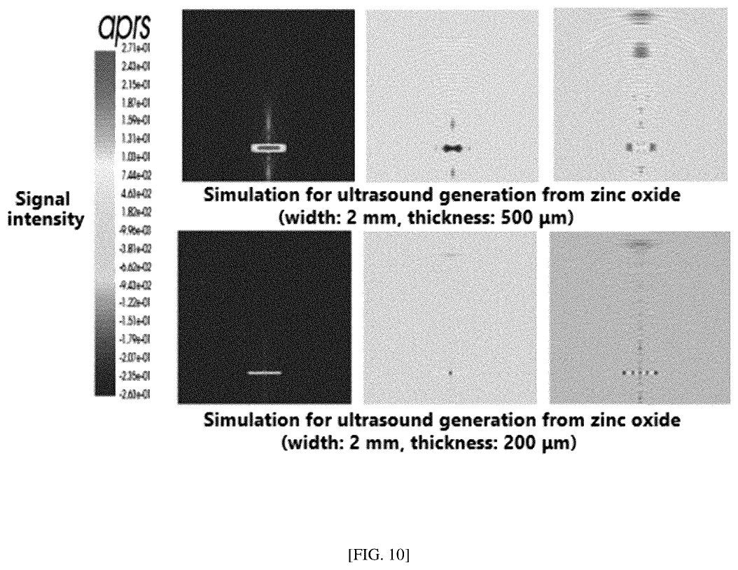

The experimental setting designed in Experimental Example 4 was used to verify the ultrasound generation from the degradable ultrasonic transducers fabricated in Experimental Example 3 and the piezoelectric effect of the degradable ultrasonic transducers. The results are shown in FIGS. 8 and 9. FIG. 8 shows data verifying the piezoelectric effect of the ultrasonic transducer using the Rochelle salt crystal as a piezoelectric element in Evaluation Example 1 and FIG. 9 shows data verifying the piezoelectric effect of the ultrasonic transducer using the zinc oxide crystal as a piezoelectric element in Evaluation Example 1.

Referring to FIG. 8, when external ultrasonic waves were applied from the commercial ultrasonic element to the degradable ultrasonic transducer using the Rochelle salt crystal as a piezoelectric element in a medium, electrical signals from the degradable ultrasonic transducer were acquired by the ultrasonic receiver. This acquisition indicates the piezoelectric effect of the degradable ultrasonic transducer, demonstrating the ability of the degradable ultrasonic transducer to generate ultrasonic waves.

Referring to FIG. 9, when external ultrasonic waves were applied from the commercial ultrasonic element to the degradable ultrasonic transducer employing zinc oxide as a piezoelectric element in the same manner as described above, the external pressures were also converted into electrical signals by the degradable ultrasonic transducer.

### Evaluation Example 2: Results of Simulations for Ultrasound Generation from Zinc Oxide Piezoelectric Elements

The relationship between the resonant frequency generated from an ultrasonic probe and the thickness of the ultrasonic probe is given by the following equation:

$${f}_{x} = \frac{{c}_{x}}{2 {l}_{x}}$$

where fₓ is the resonant frequency (desired frequency), cₓ is the velocity of sound in the element, and lₓ is the thickness of the probe.

To prove the above equation, ultrasound generation simulations were performed on zinc oxide in degradable ultrasonic piezoelectric elements. The results are shown in FIG. 10. FIG. 10 shows the results of the simulations for ultrasound generation from ultrasonic transducers using zinc oxide crystals as piezoelectric elements. The zinc oxide piezoelectric elements used for the simulations had the same width of 2 mm and different thicknesses of 200 μm and 500 μm. The results were obtained when a voltage of 200 V was applied to the zinc oxide piezoelectric elements in body fat as a medium.

Referring to FIG. 10, the ultrasonic frequency generated varied depending on the thickness of the piezoelectric element. The ultrasonic transducer using 500 μm thick zinc oxide generated the largest signals in the 5 MHz range and the ultrasonic transducer using 200 μm thick zinc oxide generated the largest signals in the 12.5 MHz range, demonstrating the ability of these ultrasonic transducers to generate ultrasonic waves in the corresponding ranges.

In conclusion, the inventive degradable ultrasonic transducer will be able to generate an ultrasonic frequency ranging from hundreds of kHz to 100 MHz by adjusting the thickness of the piezoelectric element.

### Evaluation Example 3: Verification of Similarity of Actual Results and the Simulation Results

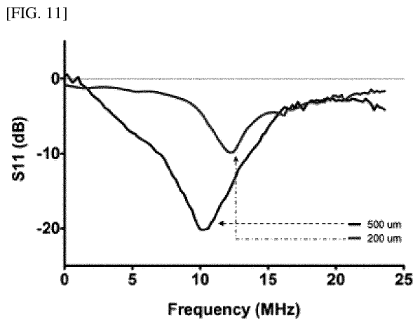

In this example, similarity of actual results and the simulation results obtained in Evaluation Example 2 was verified. To this end, the ranges where ultrasonic waves were generated from the ultrasonic transducers with different thicknesses was confirmed by determining the frequency ranges where electrical signals were converted into ultrasonic signals based on the S11 parameter. FIG. 11 shows the experimental results confirming the ranges where ultrasonic waves were generated from the ultrasonic transducers with different thicknesses using zinc oxide as a piezoelectric element.

Referring to FIG. 11, the S11 parameter conversion results confirmed that the ultrasonic transducer using 500 μm thick zinc oxide generated ultrasonic waves at a frequency of ˜10 MHz and the ultrasonic transducer using 200 μm thick zinc oxide generated ultrasonic waves at a frequency of ˜13 MHz. These results concluded that the inventive degradable ultrasonic transducer can generate ultrasonic waves in the range of 10 to 100 MHz by adjusting the thickness of the ultrasonic transducer as needed.

Although the present invention has been described herein with reference to the foregoing specific embodiments, these embodiments do not serve to limit the invention and are set forth for illustrative purposes. It will be apparent to those skilled in the art that modifications and improvements can be made without departing from the spirit and scope of the invention.

Simple modifications and changes of the present invention belong to the scope of the present invention and the specific scope of the present invention will be clearly defined by the appended claims.

### INDUSTRIAL APPLICABILITY

The specific kind and structure of the piezoelectric material used in the degradable ultrasonic transducer of the present invention ensures high piezoelectric efficiency of the ultrasonic transducer, allows the ultrasonic transducer to be naturally degraded in a use environment, and enables degradation of the ultrasonic transducer at a controlled rate. Therefore, the ultrasonic transducer of the present invention can be applied to eco-friendly electronic devices. Therefore, the present invention is considered industrially applicable.

1. A degradable ultrasonic transducer comprising a piezoelectric layer comprising a degradable piezoelectric material, a first degradable electrode arranged at one side of the piezoelectric layer, and a second degradable electrode arranged at the other side of the piezoelectric layer.

2. The degradable ultrasonic transducer according to claim 1, wherein the piezoelectric material comprises at least one selected from the group consisting of Rochelle salt, potassium dihydrogen phosphate (KDP), ammonium dihydrogen phosphate (ADP), and triglycine sulfate (TGS).

3. The degradable ultrasonic transducer according to claim 1, wherein the piezoelectric material comprises at least one selected from the group consisting of zinc oxide (ZnO) and quartz.

4. The degradable ultrasonic transducer according to claim 3, wherein the piezoelectric material is in the form of one or more nanostructures selected from the group consisting of nanorods, nanopillars, and nanowires.

5. The degradable ultrasonic transducer according to claim 1, wherein each of the first and second electrodes independently comprises at least one material selected from the group consisting of zinc (Zn), zinc (Zn)-magnesium (Mg) alloys, magnesium (Mg), molybdenum (Mo), and tungsten (W).

6. The degradable ultrasonic transducer according to claim 1, further comprising a degradable cover layer surrounding the piezoelectric layer.

7. The degradable ultrasonic transducer according to claim 6, wherein the cover layer comprises at least one selected from the group consisting of poly(lactic-co-glycolic acid) (PLGA) and poly-butanedithiol pentenoic anhydride (PBTPA).

8. The degradable ultrasonic transducer according to claim 1, further comprising a degradable matching layer arranged to face the piezoelectric layer with the first electrode interposed therebetween.

9. The degradable ultrasonic transducer according to claim 8, wherein the matching layer comprises at least one selected from the group consisting of silicon dioxide (SiO₂), zinc oxide (ZnO), and poly(lactic-co-glycolic acid) (PLGA).

10. The degradable ultrasonic transducer according to claim 1, further comprising a degradable backing layer arranged to face the piezoelectric layer with the second electrode interposed therebetween.

11. The degradable ultrasonic transducer according to claim 10, wherein the backing layer comprises at least one metal powder selected from the group consisting of zinc (Zn) powder, magnesium (Mg) powder, and tungsten (W) powder and at least one material selected from the group consisting of natural wax mixtures, poly(lactic-co-glycolic acid) (PLGA), poly-butanedithiol pentenoic anhydride (PBTPA), titanium (Ti), alumina, ceramics, and animal bones.
